(19) United States
(12) Patent Application Publication
(10) Pub. No.: US 2010/0093061 A1
(43) Pub. Date: Apr. 15, 2010

(54) Assays for Improved Fungal Strains

(76) Inventor: Elizabeth A. Bodie, San Carlos, CA (US)

Correspondence Address: DANISCO US INC.;ATTENTION: LEGAL DEPARTMENT, 925 PAGE MILL ROAD, PALO ALTO, CA, 94304, US

(21) Appl. No.: 12/519,870

(22) PCT Filed: Dec. 18, 2007
(86) PCT No.: PCT/US07/25926

§ 371 Date: Nov. 18, 2009

(51) Int. Cl. C12N 1/15

(52) U.S. Cl. 435/254.11

(57) ABSTRACT

The invention relates to methods for obtaining improved strains of filamentous ascomycete fungus, such as Trichoderma reesei, and the product of said method. The method includes contacting ascomycete cells comprising a reporter gene construct with a mutagen, wherein said reporter gene construct comprises a promoter operably linked with a reporter gene; culturing said ascomycete cells under conditions that repress activity of the promoter; and isolating said ascomycete cells that produce the reporter in amounts detectably higher than ascomycete cells which have not been contacted with the mutagen.

### 2. CROSS-REFERENCES TO RELATED APPLICATION

The present application claims benefit of and priority to U.S. Provisional Application Ser. No. 60/876,187, entitled “Assays for Improved Fungal Strains”, filed Dec. 20, 2006, incorporated herein by reference in its entirety.

### 1. GOVERNMENT SUPPORT

Portions of this work were funded by Subcontract No. ZCO-30017-01 with the National Renewable Energy Laboratory under Prime Contract No. DE-AC36-99GO10337 with the U.S. Department of Energy. Accordingly, the United States Government may have certain rights in this invention.

### 3. FIELD OF THE INVENTION

This invention relates generally to screening methods using a reporter and a repressible promoter. The invention is applicable to improving the production of a polypeptide of interest in filamentous fungi. The improved strains obtained by the process of the present invention are also provided.

### 4. BACKGROUND OF THE INVENTION

Biomass which largely consists of cellulose, hemicellulose and lignin has attracted increasing attention as an important renewable source of energy (including nutritional energy). The amount of carbon fixed by photosynthesis has been estimated to be 100×10⁹ tons per year worldwide, and half of that is contained in cellulose. If this material, or at least a significant part of it, could be converted into liquid fuel, gas and feed protein, this would constitute a significant contribution to solving the problem of recycling and conservation of resources. However, it has been difficult to develop an economically viable process of converting cellulosic material into fermentable sugars.

Currently, the most promising of the suggested processes involves the use of enzymes which are able to degrade cellulose. These enzymes which are collectively known as cellulases are produced by a number of microorganisms, including fungi (e.g. Trichoderma reesei, Humicola insolens, Fusarium oxysporum) and bacteria (e.g. Clostridium thermocellum, Cellulomonas spp., Thermonospora spp., Bacterioides spp., Microbispora bispora). The economics of the production of fermentable sugars from biomass by means of such enzymes is not yet competitive. As the limits of non-renewable resources approach, the potential of cellulose to become a major renewable energy resource is enormous. The effective utilization of cellulose through biological processes is one approach to overcoming the shortage of foods, feeds, and fuels.

Filamentous fungi and cellulolytic bacteria produce extracellular cellulase enzymes that confer on the organisms the ability to hydrolyze the β-(1,4)-linked glycosidic bonds of cellulose to produce glucose. These enzymes provide the organisms with the ability to use cellulose, the most abundant plant polysaccharide, for growth.

The filamentous fungus, Trichoderma reesei, is an efficient producer of cellulase enzymes. As such, Trichoderma reesei has been exploited for its ability to produce these enzymes, which are valuable in the production of such commodities as fuel ethanol, clothing, detergents, fibers and other products.

The cellulolytic mix of Trichoderma reesei proteins is among the best characterized cellulolytic pathways of microorganisms. The cellulases that comprise these mixes are classified into two broad categories: the endoglucanases (EG) and the cellobiohydrolases (CBH). β-glucosidase (BG) is also part of the cellulase mix of Trichoderma reesei. The fungal cellulase classifications of CBH, EG and BG can be further expanded to include multiple components within each classification. For example, multiple CBHs, EGs and BGs have been isolated from a variety of fungal sources including Trichoderma reesei which contains known genes for 2 CBHs, i.e., CBH I and CBH II, at least 5 EGs, i.e., EG I, EG II, EG III, EGIV and EGV, and at least 2 BGs, i.e., BG1 and BG2.

Trichoderma reesei has also been exploited for its ability to produce heterologous proteins. Genes encoding a desired protein can be regulated when they are operably linked to the inducible cbh1 promoter of Trichoderma reesei. Foreign polypeptides have been secreted in Trichoderma reesei as fusions with the catalytic domain plus linker region of cbh1 (Nyyssonen et al., Biotechnology 11:591-595, 1993).

Expression of the genes comprising the cellulase system is coordinated and regulated at the transcriptional level. The members of this system act synergistically, and as noted above, are necessary for the efficient hydrolysis of cellulose to soluble oligosaccharides.

Expression and production of the main cellulase genes in Trichoderma, cbh1, cbh2, eg11, and eg12, is dependent on the carbon source available for growth. The cellulase genes are tightly repressed by glucose and are induced several thousand fold by cellulose or the disaccharide sophorose. Indeed, the expression level of the major cellobiohydrolase 1 (cbh1) is up-regulated several thousand fold on media containing inducing carbon sources such as cellulose or sophorose compared with glucose containing media (Ilmen et al., App. Environ. Microbiol., 1298-1306, 1997).

Commercial scale production of cellulase enzymes is by either solid or submerged culture including batch, fed batch, and continuous flow processes. A problematic and expensive aspect of industrial cellulase production is providing the appropriate inducer to Trichoderma. As is the case for laboratory scale experiments, cellulase production on a commercial scale is induced by growing the fungus on solid cellulose or by culturing the organism in the presence of a disaccharide inducer such as lactose. Unfortunately on an industrial scale, both methods of induction have drawbacks which result in high costs being associated with cellulase production.

The production of cellulase is subject to both cellulose induction and glucose repression. Thus, a critical factor influencing the yield of cellulase enzymes or heterologous proteins under the control of an inducible promoter is the maintenance of a proper balance between cellulose substrate and glucose concentration. This balance between induction by cellulose and repression by glucose is critical for obtaining reasonable commercial yields of cellulase enzyme. Although cellulose is an effective and inexpensive inducer, controlling the glucose concentration when Trichoderma is grown on solid cellulose can be problematic. At low concentrations of cellulose, glucose production may be too slow to meet the metabolic needs of active cell growth and function. On the other hand, cellulase synthesis can be halted by glucose repression when glucose generation is faster than consumption. Thus, expensive process control schemes are required to provide slow substrate addition and monitoring of glucose concentration (Ju and Afolabi, Biotechnol. Prog., 91-97, 1999).

Presently, due to the critical importance of cellulase enzyme in the process for generating biofuels, a need clearly exists for novel methods to increase cellulase production from filamentous ascomycete fungi, e.g., Trichoderma reesei such that the cellulase enzyme can be economically available to the alternative fuel industry for their endeavors to provide technology which would reduce dependency on oil. Specifically, the need exists for the obtaining and isolating of improved fungal strains capable of increased production of cellulase enzymes.

### 5. SUMMARY OF THE INVENTION

Described herein is a method for obtaining an improved strain of filamentous fungus. The method involves the genetically engineered cells of a strain of filamentous fungus which comprises a reporter gene construct. The test cells are contacted with a mutagen to produce a population of mutant cells. The reporter gene construct in the test cells comprises an inducible or repressible promoter that is operably linked with a reporter gene. The population of mutant cells is cultured under conditions that repress the activity of the promoter, and cells that produce the reporter under a repressible condition are isolated. The conditions that repress the promoter may include pH, temperature, osmolarity, the presence of an inhibitor, the concentration of an inhibitor, or a combination of any two or more of the foregoing. In certain embodiments, the method of the invention further comprises determining the level of a polypeptide of interest produced by the isolated cells.

The invention also provides the improved strain of filamentous fungus made according to the methods of the present invention. The cells of the improved strain comprise a reporter gene construct wherein a promoter is operably linked with a reporter gene, and the cells produce the reporter under conditions that repress activity of the promoter. The improved cells are made by contacting test cells with a mutagen and isolating cells that produce a detectable amount of the reporter under repressible conditions. According to the invention, the mutagen used to generate the population of mutant cells can be ultra violet light, X-ray, gamma radiation, nitrous acid, nitrosamines, nitrosoguanidine, methyl nitrosoguanidine, and 5-bromouracil, or any combination thereof. In a specific embodiment, insertional mutagenesis is used to generate a population of mutant cells.

The invention is directed generally to filamentous fungus, and ascomycete fungi in particular. In various embodiments, the parental cell strain is of a species of Acremonium, Aspergillus, Chrysosporium, Fusarium, Gliocladium, Humicola, Myceliophthora, Mucor, Neurospora, Penicillium, Thielavia, Tolypocladium, Trichoderma, Hypocrea, or teleomorphs or synonyms thereof. In preferred embodiments, the filamentous fungus used for practicing the method of the present invention is Trichoderma reesei, Trichoderma viride, Trichoderma longibrachiatum, Trichoderma harzianum, or Trichoderma koningii.

In various embodiments, the promoter can be a catabolite repressible promoter, a temperature-sensitive promoter, or a promoter that is sensitive to changes in osmolarity. In specific embodiments, the promoter is a naturally occurring promoter that regulates the expression of cbh1, cbh2, eg1, eg2, eg3, eg5, xln1, or xln2 in Trichoderma species. In various embodiments, the reporter produced by the test cells is an enzyme. In a particular embodiment, the reporter is a fungal laccase. More specifically, the reporter can be a laccase of a Stachybotrys species.

In certain embodiments, the improved strains are Trichoderma strains that produce large amounts of cellulase enzymes. The isolated cells can produce at least about 10%, or at least about 20%, at least about 30%, at least about 60%, at least about 100%, at least about 200%, at least about 300%, at least about 400%, at least about 500%, at least about 600%, at least about 700%, at least about 800%, at least about 900%, or at least about 1000% more cellulase enzymes than the parental cells.

### 6. DESCRIPTION OF THE FIGURES

FIG. 1 is a representative example of the results after catabolite derepression screen. A positive mutant among 100,000 mutated spores is shown when grown in the presence of excess glucose and the laccase substrate ABTS.

FIG. 2 is a table depicting the screening efficiency of the laccase catabolite derepression screen and the laccase induction screen.

### 7. DETAILED DESCRIPTION OF THE INVENTION

Currently the most promising of the suggested processes to develop an economically viable process of converting cellulosic material into fermentable sugars involves the use of enzymes that are able to degrade cellulose. The filamentous fungus Trichoderma reesei is one of the most extensively studied cellulolytic organisms (reviewed e.g. by Nevalainen and Penttila, 1995, Mycota, 303-319). In industry, the cellulolytic enzymes of Trichoderma are used for many purposes including production of fuel ethanol, paper, rayon, cellophane, detergents and fibers. Thus, these enzymes are of primary importance in the production of many useful products.

Provided herein are methods for obtaining improved strains of a filamentous fungus that produce one or more polypeptide(s) of interest. Preferably, the filamentous fungus is an ascomycete. Without being bound by theory, it is believed that the mutated fungal cells described herein, in which certain aspects of the repression of gene expression is perturbed, are likely to be highly productive in making and/or secreting the gene products the expression of which are under repression.

The methods described herein generally comprises providing a strain of filamentous fungus that comprises a reporter gene construct, mutagenizing the strain, and growing the mutants under conditions that repress expression of the reporter, and identifying mutants that express the reporter, preferably in a high throughput format. More particularly, the methods described herein for obtaining an improved strain of filamentous fungus, comprising contacting test cells of a strain of filamentous ascomycete fungus comprising a reporter gene construct with a mutagen thereby producing a population of mutant cells, wherein said reporter gene construct comprises a promoter operably linked with a reporter gene, culturing said population of mutant cells under conditions that repress activity of the promoter, and isolating cells from said population of mutant cells that produce the reporter.

The reporter gene construct of the invention is designed such that expression of the reporter gene in the fungal parental cells is under the control of a promoter that responds to the repressive conditions to which the fungal cells are exposed. Many such repressible conditions are encountered by fungal cells during production of a polypeptide of interest in a biotechnological process and often these conditions adversely affect the yield of the process. For example, during production of cellulase, increasing glucose levels represses the promoter responsible for cellulase production.

In designing the reporter assays of the invention, Applicants discovered the necessity to titrate the strength of the repressible condition, for example the glucose concentration, in order to obtain at least a usable signal-to-background ratio from the reporter assay. With experimentation for the best signal-to-background ratio, one can detect a signal more accurately in the reporter assay and as a result, the detected signals are more reliable. It is thus possible to screen for improved fungal strains more effectively and efficiently than before. According to the invention, a range of catabolite concentrations, pH, temperature, and other repressible conditions are studied in the reporter assay in order to obtain a screening method that is robust enough to identify a reliable signal from the population of mutant cells.

For different repressible conditions, a different promoter can be used in building the construct. The reporter construct is introduced into the cells of a strain of filamentous fungus. Detailed description of the reporter gene constructs of the invention are provided in Section 5.1 herein below. Methods for transforming filamentous fungi are provided in Section 5.1.4.

As used herein the term “parental cells” refers to cells of a filamentous fungus into which a reportor gene construct of the invention is introduced. The term “test cells” refers to parental cells that harbor the reporter gene construct of the invention. When a batch of test cells are mutagenized, a population of mutant cells is formed, each mutant cell comprising one or more genetic mutations. The term “mutant cells” refers to test cells that were exposed to a mutagen and survived the mutagenesis. The mutagenesis step is described in detail in Section 5.2.1.

Among the population of mutant cells produced in the mutagenesis step, it is expected that some of the mutations affect mechanisms which govern directly or indirectly the regulatory control of gene expression. Some of the mechanisms affected by the mutations effectuate the cell's response in gene expression to certain repressible conditions. The inventors are particularly interested in those mutations that derepress the expression of genes which are normally tightly regulated in response to various repressible conditions. Also of interest are those mutations that generally affect the production and/or secretion of a polypeptide in the cells under normal conditions or repressible conditions. The assays of the invention are designed to identify and isolate efficiently cells comprising such mutations.

By growing the population of mutant cells under repressible conditions, mutant cells in which expression of the reporter gene is derepressed will produce the reporter and generate a signal that will lead to their identification and isolation. The conditions that repress the promoter in a reporter gene construct of the invention include, but are not limited to, pH, temperature, osmolarity, the presence of an inhibitor, the concentration of an inhibitor, or a combination of any two or more of the foregoing. The mutagenesis step also generates mutant cells in which production and/or secretion of a polypeptide is generally increased under normal or repressible conditions. Such mutant cells also produce the reporter and generate a signal that will lead to their identification and isolation. As used herein the term “candidate cells” refers to mutant cells that produce the reporter under repressible conditions as identified by screening assays of the invention. The screening assays are based on detecting a signal generated by expression of the reporter in a mutant cell against a background of mutant cells that either do not produce the reporter or produce the reporter at a much lower level or at a level that is not detectable. The candidate cells of filamentous fungus of the present invention may not respond or may respond to a lesser degree to the repressible condition than the parental cells and the test cells that were not mutagenized. The screening assays of the invention that allow identification and isolation of candidate cells are described in Section 5.2.2.

According to the invention, in instances where the promoter used to express the reporter gene is a promoter that also controls the expression of one or more native gene(s) in the parental cell, it is expected that the expression of the native gene product(s) in a candidate cell will also be derepressed when the candidate cell is placed under repressible conditions. For example, if the cbh1 promoter is used in the reporter construct, the expression of the corresponding native cellobiohydrolase gene in candidate cells of the invention are expected to be derepressed when the candidate cells are exposed to catabolites that affect the activity of cbh1 promoter. It is also expected that the production and/or secretion of other polypeptides of interest in the candidate cells can generally be improved under normal conditions and/or repressible conditions.

The present invention further provides a series of secondary assays to test the expression levels of various polypeptides of interest by the candidate cells under normal conditions and under the respective repressible conditions, and in scaled-up processes. Candidate cells that produce high levels of the polypeptide(s) of interest are isolated, propagated, and used as an improved strain for production of the polypeptides. Section 5.2.2. provides a detailed description of the secondary assays.

In various embodiments, under identical conditions, the candidate cells can produce at least about 0.1%, 0.2%, 0.5%, 1%, 2%, 2.5%, 5%, 7.5%, 10%, 15%, 20%, 25%, 30%, 40%, 50%, 100%, 200%, 400%, 600%, 800%, or 1000% more polypeptide(s) of interest than the starting parental cells and the test cells that were not contacted with a mutagen. Since it is expected that a range of expression levels may be observed, it is understood that this figure can represent the mean, median or maximum level of expression in a population of candidate cells or cells of an improved strain. As used herein, “a” or “an” means at least one, unless clearly indicated otherwise. The term “about,” as used herein, unless otherwise indicated, refers to a value that is no more than 10% above or below the value being modified by the term.

The candidate cells of the filamentous fungal strains obtained by the methods of the present invention may also possess improved growth characteristics in fermentation. In one embodiment, the improved property can be (a) increased yield of the polypeptide of interest, (b) improved growth, and/or (c) better secretion of the polypeptide of interest. In another embodiment, the improved property is increased yield of cellulase enzymes. In another embodiment, the improved property is improved growth of the cellulases-producing strain. In another embodiment, the improved property is more efficient secretion of cellulase enzymes.

The invention employs fermentation procedures and techniques for culturing fungi. Fermentation procedures for production of cellulase enzymes include solid or submerged culture, including batch, fed-batch and continuous-flow processes. Culturing is accomplished in a growth medium comprising an aqueous mineral salts medium, organic growth factors, the carbon and energy source material, molecular oxygen, and a starting inoculum of one or more particular microorganism species to be employed.

For clarity of disclosure, and not by way of limitation, the detailed description of the invention is divided into the subsections which follow.

7.1. Reporter Gene Constructs

7.1.1 Parental Cells

An objective of the invention is to obtain an improved strain of filamentous fungi that produces certain products of commercial value. In one embodiment, the parental strain is already a highly productive strain relative to a wild type strain. In another embodiment, the parental strain is not a highly productive strain relative to a wild type strain. In yet another embodiment, the parental strain is a wild type strain. The filamentous fungi of the present invention include all filamentous forms of the subdivision Eumycotina and Oomycota (see Alexopoulos, C. J. (1962), Introductory Mycology, New York: Wiley and Hawksworth et al., In, Ainsworth and Bisby's Dictionary of The Fungi, 8th edition, 1995, CAB International, University Press, Cambridge, UK). These fungi are characterized by a vegetative mycelium with a cell wall composed of chitin, cellulose, and other complex polysaccharides. The filamentous fungi of the present invention are morphologically, physiologically, and genetically distinct from yeasts. Vegetative growth by filamentous fungi is by hyphal elongation and carbon catabolism is obligately aerobic.

In the present invention, the filamentous fungal parent cell may be a cell of a species of, but not limited to, Achlya, Acremonium, Aspergillus, Cephalosporium, Chrysosporium, Cochliobolus, Endothia, Fusarium, Gliocladium, Humicola, Hypocrea, Myceliophthora, Mucor, Neurospora, Penicillium, Podospora, Pyricularia, Thielavia, Tolypocladium, and Trichoderma or teleomorphs or synonyms thereof. Known teleomorphs of Aspergillus include Eurotium, Neosartorya, and Emericella. Strains of Aspergillus and teleomorphs thereof are readily accessible to the public in a number of culture collections, such as the American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL). Known teleomorphs of Fusarium of the section Discolor include Gibberella gordonii, Gibberella cyanea, Gubberella pulicaris, and Gibberella zeae.

In one embodiment, the parental cell strain belongs to a species within the division Ascomycota. Such fungi produce spores in a distinctive type of microscopic sporangium also known as an ascus. This monophyletic grouping was formerly known as the Ascomycetes and is a significant and successful group of organisms, accounting for some 75% of all described fungi. Ascomycete(s) reproduce sexually by asci producing ascospores. Mycelium is a network of hyphae produced by all filamentous fungi. The mycelium is the vegetative part of the fungus that is involved in resource capture, feeding, and often mating. It is immersed in the substrate and is sometimes called a thallus. (see also Talbot, Molecular and Cellular Biology of Filamentous Fungi, pages 1-59, 2001).

In a preferred embodiment of the invention, the filamentous fungal parent cell is of the Trichoderma species, e.g., Trichoderma longibrachiatum, Trichoderma viride e.g., ATCC 32098 and 32086, Hypocrea jecorina, Trichoderma koningii, Trichoderma harzianum. In a more preferred embodiment, the filamentous fungus is Trichoderma reesei, e.g. NRRL 15709, ATCC 13631, 56764, 56765, 56466, 56767. For example, the T. longibrachiatum cellulase over-producing strain such as RL-P37 can be used, as described by Sheir-Neiss et al. in Appl. Microbiol. Biotechnology, 20 (1984) pp. 46-53, since this strain secretes elevated amounts of cellulase enzymes. This strain can be used according to the teachings of the present invention in order to improve even further the strain's cellulase enzyme production capability.

In another embodiment, the filamentous fungal parent cell is an Aspergillus cell. In a related embodiment, the filamentous fungal parent cell is an Aspergillus oryzae, Aspergillus niger (Kelly and Hynes (1985) EMBO J. 4, 475-479), Aspergillus awamori e.g., NRRL 3112, ATCC 22342, ATCC 44733, ATCC 14331, and strain UVK 143f, Aspergillus aculeatus, Aspergillus foetidus, Aspergillus nidulans (Yelton, M., et al. (1984) Proc. Natl. Acad. Sci. USA, 81, 1470-1474; Mullaney, E. J. et al. (1985) Mol. Gen. Genet. 199, 37-45; John, and Peberdy (1984) Enzyme Microb. Technol. 6, 386-389; Tilburn, et al. (1982) Gene 26, 205-221; Ballance, D. J. et al., (1983) Biochem. Biophys. Res. Comm. 112, 284-289; Johnston, I. L. et al. (1985) EMBO J. 4, 1307-1311), or Aspergillus japonicus, Aspergillus oryzae, e.g., ATCC 11490, cell. In yet another embodiment, the filamentous fungal parent cell is an Acremonium cell.

In yet another embodiment, the filamentous fungal parent cell is a Fusarium cell, e.g., a Fusarium cell of the section Elegans or of the section Discolor. In a related embodiment, the filamentous fungal parent cell is a Fusarium strain of the section Discolor (also known as section Fusarium). For example, the filamentous fungal parent cell may be a Fusarium bactridioides, Fusarium cerealis, Fusarium crookwellense, Fusarium culmorum, Fusarium graminearum, Fusarium graminum, Fusarium heterosporum, Fusarium negundi, Fusarium reticulatum, Fusarium roseum, Fusarium sambucinum, Fusarium sarcochroum, Fusarium sulphureum, Fusarium toruloseum, Fusarium trichothecioides, or Fusarium venenatum cell. In another embodiment, the filamentous fungal parent cell is a Fusarium venenatum cell (Nirenberg sp. nov.). In another embodiment, the filamentous fungal parent cell is a Fusarium strain of the section Elegans, e.g., Fusarium oxysporum.

In yet another embodiment, the filamentous fungal parent cell is a Humicola cell, such as but not limited to a cell of Humicola insolens or Humicola lanuginosa. In yet another embodiment, the filamentous fungal parent cell is a Myceliophthora cell, such as but not limited to a cell of Myceliophthora thernophilum. In yet another embodiment, the filamentous fungal parent cell is a Mucor cell such as but not limited to a cell of Mucor miehei.

In yet another embodiment, the filamentous fungal parent cell is a Neurospora cell. In a related embodiment, the filamentous fungal parent cell is a Neurospora crassa cell (Case, M. E. et al. (1979) Proc. Natl. Acad. Sci. USA, 76, 5259-5263; Lambowitz U.S. Pat. No. 4,486,553; Kinsey, J. A. and J. A. Rambosek (1984) Molecular and Cellular Biology 4, 117-122; Bull, J. H. and J. C. Wooton (1984) Nature 310, 701-704).

In yet another embodiment, the filamentous fungal parent cell is a Penicillium cell, such as but not limited to a cell of Penicillium purpurogenum. In another embodiment, the filamentous fungal parent cell is a Thielavia cell such as but not limited to a cell of Thielavia terrestris. In yet another embodiment, the filamentous fungal parent cell is a Tolypocladium cell, or a C. lucknowense cell.

7.1.2 Promoters

As described above, the present invention relates to improved strains of filamentous fungi wherein the expression of certain repressible genes are derepressed, i.e., the expression of the repressible genes is unresponsive to a repressible condition, or is less responsive relative to a wild type strain or standard strain in the presence of a repressible condition. For example, expression of the genes encoding cellulases is coordinated and regulated at the transcriptional level and the enzymes are necessary for the efficient hydrolysis of cellulose to soluble oligosaccharides.

In Trichoderma, expression and production of the main cellulase genes, cbh1, cbh2, eg11, and eg12, is dependent on the carbon source available for growth. The cellulase genes are tightly repressed by glucose and are induced several thousand fold by cellulose or the disaccharide sophorose. Indeed, the expression level of the major cellobiohydrolase 1 (cbh1) is up-regulated several thousand fold on media containing inducing carbon sources such as cellulose or sophorose compared with glucose containing media (Ilmen et al., App. Environ. Microbio., 1298-1306, 1997).

As used herein, the term promoter refers to a nucleic acid sequence that functions to direct transcription of a downstream gene. A promoter may include necessary nucleic acid sequences near the start site of transcription, such as, in the case of a polymerase II type promoter, a TATA element. The promoter together with other transcriptional and translational regulatory nucleic acid sequences, collectively referred to as regulatory sequences controls the expression of a gene. In general, the regulatory sequences include, but are not limited to, promoter sequences, ribosomal binding sites, transcriptional start and stop sequences, translational start and stop sequences, and enhancer or activator sequences. The regulatory sequences will generally be appropriate to and recognized by the filamentous fungal parental cell in which the downstream gene is being expressed.

A constitutive promoter is a promoter that is active under most environmental and developmental conditions. An inducible or repressible promoter is a promoter that is active under environmental or developmental regulation. Promoters can be inducible or repressible by changes in environment factors such as, but not limited to, temperature, pH, osmolarity, the presence of heavy metal, the concentration of an inhibitor, stress, or a combination of the foregoing, as is known in the art. Promoters can be inducible or repressible by metabolic factors, such as the level of certain carbon sources, the level of certain energy sources, the level of certain catabolites, or a combination of the foregoing, as is known in the art.

Examples of promoters include CBH1, CBH2, Eg1, Eg2, Eg3, Eg4, Eg5, xyl 1, and xyl 2, repressible acid phosphatase gene (phoA) promoter of P. chrysogenum (see Graessle et al. Applied and Environmental Microbiology (1997), 63(2), 753-756), glucose-repressible PCK1 promoter (see Leuker et al. Gene (1997), 192(2), 235-240), maltose-inducible, glucose-repressible MRP1 promoter (see Munro et al. Molecular Microbiology (2001), 39(5), 1414-1426), methionine-repressible MET3 promoter (see Liu et al. Eukaryotic Cell (2006), 5(4), 638-649).

In one embodiment of the invention, the promoter in the reporter gene construct is a temperature-sensitive promoter. Preferably, the activity of the temperature-sensitive promoter is repressed by elevated temperature. In another embodiment, the promoter is a catabolite-repressed promoter. In yet another embodiment, the promoter is repressed by changes in osmolarity. In certain embodiments, the promoter is inducible or repressible by the levels of polysaccharides, disaccharides, or monosaccharides.

An example of an inducible promoter useful in the present invention is the cbh 1 promoter of Trichodmera reesei, the nucleotide sequence of which is deposited in GenBank under Accession Number D86235. The practice of the invention is not constrained by the choice of promoter in the reporter gene construct. However, the choice of promoter should match the inducible or repressible factor(s) used in the screening assay. Other exemplary promoters are promoters involved in the regulation of genes encoding cellulase enzymes, such as, but not limited to, cbh2, eg1, eg2, eg3, eg5, xln1 and xln2.

In one embodiment, the temperature sensitive promoter used can be a Trichoderma cellulase promoter. Preferably, the optimal temperature for expression is 23-28° C., and at temperatures above 30-37° C., expression decreases. In a related embodiment, a temperature sensitive promoter is used to express laccase and the screening is preformed at the temperature where laccase expression is minimal or completely turned off.

Accordingly, in one aspect, the invention is directed to methods for obtaining improved strains of filamentous fungus, which is unresponsive or less responsive to repressible factors such as glucose, resulting in the derepression of the production of cellulase enzymes. In particular, Applicants discovered the necessity to titrate the strength of the repressible condition according to the promoter employed in order to obtain a usable signal-to-background ratio from the reporter assay.

The term “operably linked” refers to a functional linkage between a nucleic acid expression regulatory sequence (such as a promoter, or array of transcription factor binding sites) and a second nucleic acid sequence, wherein the regulatory sequence directs transcription of the nucleic acid corresponding to the second sequence. A nucleic acid is “operably linked” when it is placed into a functional relationship with another nucleic acid sequence. For example, DNA encoding a secretory leader, i.e., a signal peptide, is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient matched restriction sites. If such sites do not exist, the synthetic oligonucleotide adaptors or linkers are used in accordance with conventional practice.

As used herein, the term “gene” means the segment of DNA involved in producing a polypeptide chain, that may or may not include regions preceding and following the coding region, e.g. 5′ untranslated (5′ UTR) or “leader” sequences and 3′ UTR or “trailer” sequences, as well as intervening sequences (introns) between individual coding segments (exons). As described in detail in the following section, the gene encodes a reporter that generates a detectable signal for use in the methods of the invention. In other embodiments, the gene encodes commercially important industrial proteins or peptides, such as enzymes, e.g., proteases, mannanases, xylanases, amylases, glucoamylases, cellulases, oxidases and lipases. The gene of interest may be a naturally occurring gene, a mutated gene or a synthetic gene.

In addition to a promoter sequence, the reporter gene construct may contain a transcription termination region downstream of the reporter gene to provide for efficient termination. The termination region may be obtained from the same gene as the promoter sequence or may be obtained from a different gene. Although any fungal terminator is likely to be functional in the present invention, preferred terminators include: the terminator from Aspergillus nidulans trpC gene (Yelton, M. et al. (1984) Proc. Natl. Acad. Sci. USA 81:1470-1474, Mullaney, E. J. et al. (1985) MGG 199:37-45), the Aspergillus awamori or Aspergillus niger glucoamylase genes (Nunberg, J. H. et al. (1984) Mol. Cell. Biol. 4:2306, Boel, E. et al. (1984) EMBO J. 3:1581-1585) and the Mucor miehei carboxylprotease gene (EPO Publication No. 0 215 594).

7.1.3 Reporters

The invention provides a reporter gene in the reporter gene construct to monitor the activity of the upstream inducible or repressible promoter. When the reporter gene is expressed, a reporter is produced which generates a detectable signal in the cell harboring the reporter gene construct. Many reporter genes are known in the art and can be used in the present invention. Preferably, the signal generated by the reporter is easily detectable and amenable to screening in a high-throughput format. To minimize background signal, it is preferable that the reporter is not normally expressed in the parental cell. Some reporters, such as enzymes, require the use of accessory molecules to generate a signal. Some reporters, such as green fluorescent protein, do not require accessory molecules to signal.

In one embodiment of the present invention, laccase is used as a reporter. Laccases (benzenediol:oxygen oxidoreductases) are multi-copper containing enzymes that catalyze the oxidation of phenolics. Laccase-mediated oxidations result in the production of aryloxy-radical intermediates from suitable phenolic substrate; the ultimate coupling of the intermediates so produced provides a combination of dimeric, oligomeric, and polymeric reaction products.

Laccase exhibits a wide range of substrate specificity, and each different fungal laccase usually differs quantitatively from others in its ability to oxidize phenolic substrates under different conditions, such as pH. Procedures for determining laccase activity are known in the art and include, e.g., the oxidation of the substrate 2,2′-azinobis-(3-ethybenzthiazoline-6-sulfonic acid (“ABTS”) (see, Childs et al., 1975, Biochemical Journal 145:93-103) or syringaldazine (see, Bauer et al., 1971, Analytical Chemistry 43: 421-425), or the use of 2,6 dimethoxyphenol (see Haars et al., 1981, European Journal of Forest Pathology, 11(1-2), 67-76.) or guaiacol (see Setti et al., 1999, Enzyme and Microbial Technology, 25(3-5), 285-289.)

The source of a laccase gene for the present invention may be a plant, microbial, insect, or mammalian laccase. In a preferred embodiment, the laccase(s) is a fungal laccase. For example, the laccase(s) may be a filamentous fungal laccase such as a laccase of an Acremonium, Agaricus, Antrodiella, Armillaria, Aspergillus, Aureobasidium, Bjerkandera, Cerrena, Chaetomium, Chrysosporium, Coprinus, Cryptococcus, Cryphonectria, Curvularia, Cyathus, Daedalea, Filibasidium, Fomes, Fusarium, Geotrichum, Halosarpheia, Humicola, Hypocrea, Lactarius, Lentinus, Magnaporthe, Monilia, Monocillium, Mucor, Myceliophthora, Neocallimastix, Neurospora, Paecilomyces, Panus, Penicillium, Phanerochaete, Phellinus, Phlebia, Pholiota Piromyces, Pleurotus, Podospora, Pycnoporus, Pyricularia, Rigidoporus, Rhizoctonia, Schizophyllum, Sclerotium, Scytalidium Sordaria, Sporotrichum, Stagonospora, Talaromyces, Thermoascus, Thielavia, Tolypocladium, Trametes (Polyporus) or Trichoderma species, or a yeast laccase from Candida, Kluyveromyces, Pichia, Saccharomyces, Schizosaccharomyces, or Yarrowia species. More specifically, the laccase can be a laccase of Coprinus cinereus, Myceliophthora thermophila, Trametes villosa (Polyporus pinsitus), Rhizoctonia solani, or Scytalidium thermophilum laccase.

In another embodiment, the laccase(s) is a plant laccase. For example, the laccase(s) may be a lacquer, mango, mung bean, peach, pine, prune, or sycamore laccase. In yet another embodiment, the laccase(s) is an insect laccase. For example, the laccase(s) may be a Bombyx, Calliphora, Diploptera, Drosophila, Lucilia, Manduca, Musca, Oryctes, Papilio, Phorma, Rhodnius, Sarcophaga, Schistocerca, or Tenebrio laccase.

In yet another embodiment, the laccase(s) is preferably a bacterial laccase. For example, the laccase(s) may be a laccase of Acer, Acetobacter, Acinetobacter, Agrobacterium, Alcaligenes, Arthrobacter, Azotobacter, Bacillus, Comamonas, Clostridium, Gluconobacter, Halobacterium, Mycobacterium, Rhizobium, Salmonella, Serratia, Streptomyces, E. coli, Pseudomonas, Wolinella, or a methylotrophic bacteria. More specifically, the laccase is a Azospirillum lipoferum laccase.

In other embodiments, the reporter is not a laccase. For example, the reporter can be a luciferase. Luciferases are enzymes that emit light in the presence of oxygen and a substrate (luciferin) and which have been used for real-time, low-light imaging of gene expression in cell cultures, individual cells, whole organisms, and transgenic organisms (reviewed by Greer & Szalay, 2002, Luminescence 17:43-74).

As used herein, the term “luciferase” is intended to embrace all luciferases, or recombinant enzymes derived from luciferases which have luciferase activity. The luciferase genes from fireflies have been well characterized, for example, from the Photinus and Luciola species (see, e.g., International Patent Publication No. WO 95/25798 for Photinus pyralis, European Patent Application No. EP 0 524 448 for Luciola cruciata and Luciola lateralis, and Devine et al., 1993, Biochim. Biophys. Acta 1173:121-132 for Luciola mingrelica). Other eucaryotic luciferase genes include, but are not limited to, the sea panzy (Renilla reniformis, see, e.g., Lorenz et al., 1991, PNAS 88:4438-4442), and the glow worm (Lampyris noctiluca, see e.g., Sula-Newby et al., 1996, Biochem J. 313:761-767). Bacterial luciferin-luciferase systems include, but are not limited to, the bacterial lux genes of terrestrial Photorhabdus luminescens (see, e.g., Manukhov et al., 2000, Genetika 36:322-30) and marine bacteria Vibrio fischeri and Vibrio harveyi (see, e.g., Miyamoto et al., 1988, J. Biol. Chem. 263:13393-9, and Cohn et al., 1983, PNAS 80:120-3, respectively). The luciferases encompassed by the present invention also includes the mutant luciferases described in U.S. Pat. No. 6,265,177.

In yet another embodiment, β-galactosidase (“β-gal”) is used as a reporter. β-gal is an enzyme that catalyzes the hydrolysis of β-galactosides (e.g., lactose) as well as galactoside analogs (e.g., o-nitrophenyl-β-D-galactopyranoside (“ONPG”) and chlorophenol red-β-D-galactopyranoside (“CPRG”)) (see, e.g., Nielsen et al., 1983 PNAS 80:5198-5202; Eustice et al., 1991, Biotechniques 11:739-742; and Henderson et al., 1986, Clin. Chem. 32:1637-1641). As used herein, the term “beta galactosidase” or “β-gal” is intended to embrace all β-gals, including lacZ gene products, or recombinant enzymes derived from β-gals which have β-gal activity. The β-gal gene functions well as a reporter gene because the protein product is extremely stable, resistant to proteolytic degradation in cellular lysates, and easily assayed. In an embodiment where ONPG is the substrate, β-gal activity can be quantitated with a spectrophotometer or microplate reader to determine the amount of ONPG converted at 420 nm. In an embodiment when CPRG is the substrate, β-gal activity can be quantitated with a spectrophotometer or microplate reader to determine the amount of CPRG converted at 570 to 595 nm. In yet another embodiment, the β-gal activity can be visually ascertained by plating bacterial cells transformed with a β-gal construct onto plates containing Xgal and IPTG. Colonies that are dark blue indicate the presence of high β-gal activity and colonies that are varying shades of blue indicate varying levels of β-gal activity.

In yet another embodiment, secreted alkaline phosphatase (“SEAP”) is used as a reporter gene. SEAP enzyme is a truncated form of alkaline phosphatase, in which the cleavage of the transmembrane domain of the protein allows it to be secreted from the cells into the surrounding media. In a preferred embodiment, the alkaline phosphatase is isolated from human placenta. As used herein, the term “secreted alkaline phosphatase” or “SEAP” is intended to embrace all SEAP or recombinant enzymes derived from SEAP which have alkaline phosphatase activity. SEAP activity can be detected by a variety of methods including, but not limited to, measurement of catalysis of a fluorescent substrate, immunoprecipitation, HPLC, and radiometric detection. The luminescent method is preferred due to its increased sensitivity over other detection methods.

Green fluorescent protein (“GFP”) is an example of a reporter that does not require accessory molecules to generate a signal. The invention contemplates the use of any fluorescent protein as a reporter. As used herein, the term “green fluorescent protein” or “GFP” is intended to embrace all GFPs (including the various forms of GFPs which exhibit colors other than green), or recombinant enzymes derived from GFPs which have GFP activity. Naturally occurring GFP is a 238 amino acid protein with amino acids 65 to 67 involved in the formation of the chromophore which does not require additional substrates or cofactors to fluoresce (see, e.g., Prasher et al., 1992, Gene 111:229-233; Yang et al., 1996, Nature Biotechnol. 14:1252-1256; and Cody et al., 1993, Biochemistry 32:1212-1218). The native gene for GFP was cloned from the bioluminescent jellyfish Aequorea victoria (see, e.g., Morin et al., 1972, J. Cell Physiol. 77:313-318). Wild type GFP has a major excitation peak at 395 nm and a minor excitation peak at 470 nm. The absorption peak at 470 nm allows the monitoring of GFP levels using standard fluorescein isothiocyanate (FITC) filter sets. Mutants of the GFP gene have been found useful to enhance expression and to modify excitation and fluorescence. For example, mutant GFPs with alanine, glycine, isoleucine, or threonine substituted for serine at position 65 result in mutant GFPs with shifts in excitation maxima and greater fluorescence than wild type protein when excited at 488 nm (see, e.g., Heim et al., 1995, Nature 373:663-664; U.S. Pat. No. 5,625,048; Delagrave et al., 1995, Biotechnology 13:151-154; Cormack et al., 1996, Gene 173:33-38; and Cramer et al., 1996, Nature Biotechnol. 14:315-319). The ability to excite GFP at 488 nm permits the use of GFP with standard fluorescence activated cell sorting (“FACS”) equipment. In another embodiment, GFPs are isolated from organisms other than the jellyfish, such as, but not limited to, the sea pansy, Renilla reriformis.

Techniques for labeling cells with GFP in general are described in U.S. Pat. Nos. 5,491,084 and 5,804,387; Chalfie et al., 1994, Science 263:802-805; Heim et al., 1994, PNAS 91:12501-12504; Morise et al., 1974, Biochemistry 13:2656-2662; Ward et al., 1980, Photochem. Photobiol. 31:611-615; Rizzuto et al., 1995, Curr. Biology 5:635-642; and Kaether & Gerdes, 1995, FEBS Lett. 369:267-271. The expression of GFPs in E. coli and C. elegans is described in U.S. Pat. No. 6,251,384.

7.1.4 Method for Constructing Test Cells

The present invention involves expression of a homologous or heterologous gene under control of an inducible or repressible promoter in filamentous fungi. Routine techniques in the field of recombinant genetics are applicable. Basic texts disclosing the general techniques used in this invention include Sambrook et al., Molecular Cloning, A Laboratory Manual (2nd ed. 1989); Kriegler, Gene Transfer and Expression: A Laboratory Manual (1990); Ausubel et al., eds., Current Protocols in Molecular Biology (1994); and Talbot, Molecular and Cellular Biology of Filamentous Fungi, pages 1-59, (2001)).

A “polypeptide of interest” refers to the polypeptide expressed and secreted by the parental cell. The polypeptide of interest may be either homologous or heterologous to the host. A polypeptide of interest may be a secreted polypeptide particularly an enzyme which is selected from amylolytic enzymes, proteolytic enzymes, cellulolytic enzymes, oxido-reductase enzymes and plant wall degrading enzymes. Examples of these enzymes include amylases, proteases, xylanases, lipases, laccases, phenol oxidases, oxidases, cutinases, cellulases, hemicellulases, esterases, perioxidases, catalases, glucose oxidases, phytases, pectinases, glucosidases, isomerases, transferases, galactosidases and chitinases. In a preferred embodiment the secreted polypeptide is a cellulolytic enzyme.

The term “heterologous” when used with reference to portions of a nucleic acid indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous protein will often refer to two or more subsequences in a polypeptide that are not found in the same relationship to each other in nature (e.g., a fusion protein).

In one embodiment, the filamentous fungus is, but not limited to, Trichoderma reesei and the expressed polypeptide(s) of interest are, but not limited to, cellulase enzymes. In another embodiment, the improved filamentous ascomycete strain is derived from a starting parental strain which is any Trichoderma sp. strain.

Heterologous genes comprising the cellulase gene promoter sequences of filamentous fungi are typically cloned into intermediate vectors before transformation into Trichoderma reesei cells for replication and/or expression. These intermediate vectors are typically prokaryotic vectors, e.g., plasmids, or shuttle vectors.

To obtain expression of a reporter gene, the heterologous reporter gene is preferably positioned about the same distance from the promoter as is in the naturally occurring gene that is downstream. Some variation in this distance can be accommodated without loss of promoter function. Those skilled in the art are aware that a natural promoter can be modified by replacement, substitution, addition or elimination of one or more nucleotides without changing its function. The practice of the invention encompasses and is not constrained by such alterations to the promoter.

The reporter gene construct typically contains a transcription unit that contains all the additional elements required for the expression of the reporter gene sequence. A typical construct thus contains a promoter operably linked to the heterologous reporter gene sequence and signals required for efficient polyadenylation of the transcript, ribosome binding sites, and translation termination. Additional elements of the construct may include enhancers and, if genomic DNA is used as the reporter gene, introns with functional splice donor and acceptor sites.

The particular vector used to transport the genetic information into the cell is not particularly critical. The elements that are typically included in expression vectors also include a replicon, a gene encoding antibiotic resistance to permit selection of bacteria that harbor recombinant plasmids, and unique restriction sites in nonessential regions of the plasmid to allow insertion of heterologous sequences. The particular antibiotic resistance gene chosen is not critical, any of the many resistance genes known in the art are suitable.

The vector may be any vector which can be conveniently subjected to recombinant DNA procedures and can bring about the expression of the nucleic acid sequence encoding the reporter. The choice of the vector will typically depend on the compatibility of the vector with the parental cell into which the vector is to be introduced. The vectors may be linear or closed circular plasmids. The vectors may remain episomal in the parental cell. The vectors may contain an element(s) that permits stable integration of the vector into the parental cell genome. For integration, the vector may rely on the nucleic acid sequence in the vector for stable integration of the vector into the genome by homologous or nonhomologous recombination. Alternatively, the vector may contain additional nucleic acid sequences for directing integration by homologous recombination into the genome of the parental cell. The additional nucleic acid sequences enable the vector to be integrated into the parental cell genome at a precise location(s) in the chromosome(s).

The vectors preferably contain one or more selectable markers which permit easy selection of transformed cells. A selectable marker is a gene the product of which provides for biocide resistance, resistance to heavy metals, prototrophy to auxotrophs, and the like. A selectable marker for use in a filamentous fungal parental cell may be selected from the group including, but not limited to, amdS (acetamidase), argB (ornithine carbamoyltransferase), bar (phosphinothricin acetyltransferase), hygB (hygromycin phosphotransferase), niaD (nitrate reductase), pyrG (orotidine-5′-phosphate decarboxylase), sC (sulfate adenyltransferase), trpC (anthranilate synthase), and glufosinate resistance markers, as well as equivalents from other species. Furthermore, selection may be accomplished by co-transformation, e.g., as described in WO 91/17243, where the selectable marker is on a separate vector. The mutant cell is preferably transformed with a vector comprising the nucleic acid construct followed by integration of the vector into the parental chromosome.

Any of the well-known procedures for introducing foreign nucleotide sequences into parental cells may be used. These include the use of calcium phosphate transfection, polybrene, protoplast fusion, electroporation, biolistics, liposomes, microinjection, plasma vectors, viral vectors and any of the other well known methods for introducing cloned genomic DNA, cDNA, synthetic DNA or other foreign genetic material into a parental cell (see, e.g., Sambrook et al., supra). It is only necessary that the particular genetic engineering procedure used be capable of successfully introducing at least one copy of the reporter gene construct into the parental cell.

A preferred method is Agrobacterium-mediated transfection as described in De Groot et al., 1998, Nat. Biotechnol. 16(9):839-42 and U.S. Pat. No. 6,255,115. By transforming cells with Agrobacterium tumefaciens, one copy of the reporter gene is inserted, thereby providing for more effective detection of improved fungal strains because the background level of the reporter is kept low.

Transformation is a means for introducing a nucleic acid construct into a parental cell so that the construct is maintained as a chromosomal integrant. Integration is generally considered to be an advantage as the nucleic acid sequence encoding the heterologous polypeptide is more likely to be stably maintained in the cell. Integration of the vector into the parental chromosome occurs by homologous or non-homologous recombination. Transformation is achieved using those techniques adapted for the fungal host being used, many of which are well known in the art. Several transformation techniques have been developed to transform filamentous fungi. For review articles on the transformation of fungi, see “Transformation in Fungi” by John R. S. Fincham published in Microbiological Reviews (March 1989) 148-170, which gives an outline of the possible transformation methods for fungi, i.e. both yeasts and moulds; “Genetic engineering of filamentous fungi” by Timberlake, W. E. and Marshall, M. A. Science 244 (1989) 1313-1317; and “Transformation” by David B. Finkelstein (Chapter 6 in the book “Biotechnology of Filamentous Fungi, Technology and Products” (1992) 113-156, edited by Finkelstein and Ball). Suitable procedures for transformation of Aspergillus cells are described in EP 238 023, Christensen et al., 1988, Bio/Technology 6:1419-1422; and Yelton et al., 1984, Proc. Natl. Acad. Sci. USA 81:1470-1474. A suitable method of transforming Fusarium species is described by Malardier et al., 1989, Gene 78:147-156 or in copending U.S. Ser. No. 08/269,449.

The methods of transformation of the present invention may result in the stable integration of all or part of the transformation vector into the genome of the filamentous fungus. However, transformation resulting in the maintenance of a self-replicating extra-chromosomal transformation vector is also contemplated.

Many standard methods can be used to produce Trichoderma reesei cell lines that express large quantities of the polypeptide of interest. Some of the published methods for the introduction of DNA constructs into cellulase-producing strains of Trichoderma include Lorito et al., 1993, Curr. Genet. 24: 349-356; Goldman et al., 1990, Curr. Genet. 17:169-174; Penftila, et al., 1987, Gene 6: 155-164; for Aspergillus, Yelton et al., 1984, Proc. Natl. Acad. Sci. USA 81: 1470-1474; for Fusarium, Bajar et al., 1991, Proc. Natl. Acad. Sci. USA 88: 8202-8212; for Streptomyces, Hopwood et al., 1985, The John Innes Foundation, Norwich, UK; and for Bacillus, Brigidi et al., 1990, FEMS Microbiol. Lett. 55: 135-138.

A selectable marker must first be chosen so as to enable detection of the transformed fungus. Any selectable marker gene which is expressed in Trichoderma sp. can be used in the present invention so that its presence in the transformants will not materially affect the properties thereof. The selectable marker can be a gene which encodes an assayable product. The selectable marker may be a functional copy of a Trichoderma sp. gene which if lacking in the parental strain results in the parental strain displaying an auxotrophic phenotype.

The parental strains used could be derivatives of Trichoderma sp. which lack or have a nonfunctional gene or genes corresponding to the selectable marker chosen. For example, if the selectable marker of pyr4 is chosen, then a specific pyr derivative strain is used as a recipient in the transformation procedure. Other examples of selectable markers that can be used in the present invention include the Trichoderma sp. genes equivalent to the Aspergillus nidulans genes argB, trpC, niaD and the like. The corresponding recipient strain must therefore be a derivative strain such as argB-, trpC-, niaD-, and the like.

7.2 Methods of the Invention

The present invention teaches a method of obtaining an improved strain of filamentous ascomycete fungus. In order to obtain an improved strain of filamentous ascomycete fungus, test cells of a strain of filamentous fungus comprising a reporter gene construct are contacted with a mutagen thereby producing a population of mutant cells, wherein the reporter gene construct comprises a promoter operably linked with a reporter gene. Subsequently, the population of mutant cells are cultured under conditions that repress activity of the promoter, and the cells that produce the reporter are isolated from the population of mutant cells. In one embodiment, before the contacting step, the method of the present invention includes the step of providing test cells of a strain of filamentous fungus comprising a reporter gene construct.

7.2.1 Mutagenesis

The parent cell may be mutagenized by methods known in the art. For example, mutagenesis of the parent cell can be achieved by irradiation, e.g., UV, X-ray, or gamma radiation of the parent cell. Furthermore, mutagenesis can be obtained by treatment with chemical mutagens, e.g., nitrous acid, nitrosamines, methyl nitrosoguanidine, and base analogues such as 5-bromouracil. In one embodiment, the mutagen is applied to spores of the parent strain, and the surviving spores are plated out on a solid medium. In another embodiment, insertional mutagenesis is used using restriction enzyme-mediated integration (“REMI”) or Agrobacterium-mediated transformation. Insertional mutagenesis is used to generate a population of mutant cells (Parinov et al., 2000, Current Opinion in Biotechnology, 11, 157-161; Seong et al., 2005, Phytopathology, 95(7), 744-750; Kuspa, 2006, Methods in Molecular Biology, 346, 201-226). In other embodiments, other forms of the fungal organism are used in the mutagenesis step (see Fiedurek et al., 1997, Enzyme and Microbial Technology 20(5), 344-347; and Gerhardt et al. 1994, American Society for microbiology, 297-316.

7.2.2 Reporter Assays

In one aspect of the invention, a method is provided that involves growing a population of mutants filamentous fungal cells under conditions that repress expression of a reporter, and identifying mutants that express the reporter. As described above, the reporter gene construct in the mutant cells is designed such that expression of the reporter is under the control of a promoter that responds to the repressive conditions to which the cells are exposed. Mechanisms that govern directly or indirectly the regulatory control of gene expression is expected to be affected by the mutations present in a population of mutagenized test cells.

The growth conditions and the assays described herein are designed to identify cells with those mutations that derepress the expression of genes which are normally tightly regulated in response to various repressible conditions, and those mutations that generally affect the production and/or secretion of a polypeptide in the cells under normal conditions or repressible conditions.

By growing the population of mutant cells under repressible conditions, mutant cells in which expression of the reporter gene is derepressed will produce the reporter and generate a signal that will lead to their identification and isolation. It is generally recommended to titrate the strength of the repressible condition to obtain at least a usable signal-to-background ratio from the reporter assay. The conditions that repress a promoter in a reporter gene construct of the invention include, but are not limited to pH, temperature, osmolarity, the presence of an inhibitor, the concentration of an inhibitor, or a combination of any two or more of the foregoing. For example, it may be required to test a range of catabolite concentrations, such as different glucose concentrations or a range of pH or temperature, before the reporter assay is robust enough to identify a reliable signal from the population of mutant cells. As used herein the term “candidate cells” refers to mutant cells that produce the reporter under repressible conditions as identified by screening assays of the invention.

The screening assays are based on detecting a signal generated by expression of the reporter in a mutant cell against a background of mutant cells that either do not produce the reporter or produce the reporter at a much lower level or at a level that is not detectable. The inventor discovered the necessity to adjust various aspects of the repressible condition being applied, for example the absolute level of a catabolite in the growth medium, or the relative concentrations of several catabolites in the growth medium, in order to obtain first a detectable signal, and then the best signal-to-background ratio from the reporter assay.

Based on the inventor's discovery, a range of one repressible condition, for example, a range of catabolite concentrations, pH, or temperature is determined in order to obtain a screening method based on a reporter assay that is robust enough to identify a reliable signal from the population of mutant cells. Applying highly repressible conditions for too long a period may completely inhibit the generation of a detectable signal. Repressible conditions that are too permissive or transient may allow many mutant cells to produce signals that will mask the signals from the most desirable mutant cells or make the assay less efficient. Candidate cells that produce high levels of the polypeptide(s) of interest are isolated, propagated, and used as an improved strain for production of the polypeptides.

The inventor has discovered that 0.1% to 30% glucose levels repress the promoter for the time period required to differentiate the repressed strains from the nonrepressed strains, and that the duration of the assay can determine the effectiveness of the screening assay. That is, if the assay is left running past a certain point in time, the mutant cells will utilize the glucose and the promoter will turn on and express the reporter, thereby increasing the background signal and thus rendering the assay less effective. For example, 0.1%, 0.2%, 0.5%, 0.75%, 1%, 1.5%, 2%, 2.5%, 5%, 7.5%, 10%, 15%, 20%, 25%, or 30% glucose can be used in the medium. The reporter assay using the presence of glucose as a repressible condition may run for 0.5 to 24 hours, for example, 1, 2, 5, 10, 15, or 20 hours, or for 1 to 15 days, such as but not limited to 2, 4, 6, 7, 8, 10, or 12 days.

The reporter may be detected using methods known in the art for the reporter which may include for example, formation of an enzyme product, or disappearance of an enzyme substrate. Many procedures for determining the enzyme activity of a reporter are well known in the art. Depending on the physical format used to grow the filamentous fungal cells, the reporter assay is adapted so that a large number of mutant cells can be screened efficiently. In one embodiment, the fungal cells can be grown in liquid phase under repressible conditions and then spread onto a solid phase for assaying. In another embodiment, fungal spores can be germinated on a solid phase under repressible conditions and then assayed on the solid phase.

In a preferred embodiment of the present invention, laccase is used as the reporter. Laccases (benzenediol:oxygen oxidoreductases) are multi-copper containing enzymes that catalyze the oxidation of phenolics. Laccase-mediated oxidations result in the production of aryloxy-radical intermediates from suitable phenolic substrate; the ultimate coupling of the intermediates so produced provides a combination of dimeric, oligomeric, and polymeric reaction products. Laccase exhibits a wide range of substrate specificity, and each different fungal laccase usually differs only quantitatively from others in its ability to oxidize phenolic substrates under different conditions, such as pH. Procedures for determining laccase activity are known in the art and include, e.g., the oxidation of the substrate 2,2′-azinobis-(3-ethybenzthiazoline-6-sulfonic acid (“ABTS”) (see, Childs et al., 1975, Biochemical Journal 145:93-103) or syringaldazine (see, Bauer et al., 1971, Analytical Chemistry 43: 421-425) as substrate.

In a non-limiting example, the reporter is a fungal laccase, the production of which is detected on agar plates comprising the laccase substrate ABTS. Generation of laccase and hydrolysis of the substrate present in agar, colors the agar which is detectable by eye, thereby enabling identification of the strain of fungus expressing the reporter. Laccase-producing transformants are identified by the formation of a dark-green halo indicative of oxidation of the ABTS substrate. Laccase activity may also be determined by syringaldazine oxidation. Specifically, syringaldazine stock solution and laccase sample are mixed with preheated Britton-Robinson buffer solution (0.1 M boric acid-0.1 M acetic acid-0.1 M phosphoric acid adjusted to pH 5.0 with 0.5 M NaOH) and incubated at 20° C. The oxidation is monitored at 530 nm over 5 minutes and activity is expressed as syringaldazine oxidized per minute (“SOU”).

In other embodiments, 2,6-dimethoxyphenol or guaiacol can be used with a laccase reporter.

In another embodiment, luciferase is the reporter gene. Luciferases are enzymes that emit light in the presence of oxygen and a substrate (luciferin) and which have been used for real-time, low-light imaging of gene expression in cell cultures, individual cells, whole organisms, and transgenic organisms (reviewed by Greer & Szalay, 2002, Luminescence 17:43-74).

In another embodiment, beta galactosidase (“β-gal”) is used as a reporter gene. β-gal is an enzyme that catalyzes the hydrolysis of β-galactosides (e.g., lactose) as well as galactoside analogs (e.g., o-nitrophenyl-β-D-galactopyranoside (“ONPG”) and chlorophenol red-β-D-galactopyranoside (“CPRG”)) (see, e.g., Nielsen et al., 1983 PNAS 80:5198-5202; Eustice et al., 1991, Biotechniques 11:739-742; and Henderson et al., 1986, Clin. Chem. 32:1637-1641).

In another embodiment, secreted alkaline phosphatase (“SEAP”) is used as a reporter gene. SEAP enzyme is a truncated form of alkaline phosphatase, in which the cleavage of the transmembrane domain of the protein allows it to be secreted from the cells into the surrounding media. In a preferred embodiment, the alkaline phosphatase is isolated from human placenta.

A preferred detection means for secreted proteins that are enzymes such as cellulases, would be fluorescent or colorimetric enzymatic assays. Fluorescent/luminescent/color substrates for alkaline phosphatase are commercially available and such assays are easily adaptable to high throughput multiwell plate screen format. Fluorescent energy transfer based assays are used for cellulase assays. Fluorophore and quencher molecules are incorporated into two ends of the substrate of the cellulase. Upon conversion of the substrate, separation of the fluorophore and quencher allows the fluorescence to be detectable. When the secreted protein could be measure by radioactive methods, scintillation proximity technology is used. The substrate of the protein of interest is immobilized either by coating or incorporation on a solid support that contains a fluorescent material. A radioactive molecule, brought in close proximity to the solid phase by enzyme reaction, causes the fluorescent material to become excited and emit visible light. Emission of visible light forms the basis of detection of successful ligand/target interaction, and is measured by an appropriate monitoring device. An example of a scintillation proximity assay is disclosed in U.S. Pat. No. 4,568,649, issued Feb. 4, 1986. Materials for these types of assays are commercially available from Dupont NEN™ (Boston, Mass.) under the trade name FlashPlate™.

In another embodiment, green fluorescent protein (“GFP”) is the reporter. GFP has been used a reporter in filamentous fungi, see for example, Fernandez-Abalos et al., 1998, Molecular Microbiology, 27(1), 121-130; Schilde et al., 2001, Archives of Microbiology, 175(1), 1-7; and Poeggeler et al., 2003, Current Genetics, 43(1), 54-61. The signal from GFP is directly correlated with the concentration of GFP produced in the cell and is not affected by the progress of the enzymatic reaction between reporter and substrate.

The term “high throughput” refers to an assay design that allows easy analysis of multiple samples simultaneously, and/or has the capacity for incorporating robotic manipulations. Various technology known in the art can be adapted to capture the signal from a plate as an image and subject the image to analysis which facilitates the identification of a candidate cell. In a preferred embodiment, the reporter assay uses a substrate that generates a visually detectable product upon reaction with an enzymatic reporter. Another desired feature of high throughput assays is an assay design that is optimized to reduce reagent usage, or minimize the number of manipulations in order to achieve the analysis desired. Examples of assay formats for the invention include 96-well or 384-well plates, and microchannel chips used for liquid handling. It is well known by those in the art that as automation and miniaturization of growth chambers and liquid handling devices are advanced, or as improved assay devices are designed, that greater numbers of samples may be performed using the mutant cells and reporters of the present invention.

The aforementioned reporter assays are used under conditions that repress the promoter in the reporter gene construct. Under such conditions where there is repression of the promoter, lower levels of the reporters are detectable. That is, the greater the repression of the promoter by conditions including for example pH, temperature, osmolarity, the presence of an inhibitor, the concentration of an inhibitor, or a combination of any two or more of the foregoing, the lower the level of detectable reporter. The improved strain of filamentous fungus of the present invention may not respond or may respond to a lesser degree to the repressible condition than the parental cells and the test cells that were not mutagenized. As such, greater levels of reporter is expressed for the improved strains under the repressible conditions.

7.3 Uses of the Mutant Cells

The use of fungal expression systems for the production of proteins of interest is well known within the art. For example, heterologous proteins have been produced within fungal expression systems for biomass conversion, detergent applications, de-pilling of cellulase substrates and other industrial enzyme uses. The production of other heterologous proteins of interest, such as food additives or supplements, pharmaceutical compounds, antibodies, protein reagents and the like, and industrial proteins is also feasible within fungal expression systems.

The method of the present invention yield improved filamentous fungus strains which produces increased levels of a polypeptide of interest. The present invention relates to improving the expression of a polypeptide of interest, in particular a fungal polypeptide, especially a fungal enzyme.

The improved filamentous fungi obtained according to the teachings of the present invention can be used to produce enzymes such as a hydrolase, an oxidoreductase, an isomerase, a ligase, a lyase, or a transferase. More preferably, the enzyme is an aminopeptidase, an amylase, a carboxypeptidase, a catalase, a cellulase, a chitinase, a cutinase, a cyclodextrin glycosyl transferase, a deoxyribonuclease, an esterase, a glucoamylase, an alpha-galactosidase, a beta-galactosidase, an alpha-glucosidase, beta-glucosidase, a haloperoxidase, an invertase, a laccase, a lipase, a mannosidase, a mutanase, an oxidase, a pectinolytic enzyme, a peroxidase, a phenoloxidase, phytase, a proteolytic enzyme, a ribonuclease, a xylanase, or a xylose isomerase. “Cellulase,” “cellulolytic enzymes” or “cellulase enzymes” means bacterial or fungal exoglucanases or exocellobiohydrolases, and/or endoglucanases, and/or β-glucosidases. These three different types of cellulase enzymes act synergistically to convert cellulose and its derivatives to glucose.

It will be understood by those skilled in the art that the term “fungal enzymes” includes not only native fungal enzymes, but also those fungal enzymes which have been modified by amino acid substitutions, deletions, additions, or other modifications which may be made to enhance activity, thermostability, pH tolerance and the like.

The improved filamentous fungi identified according to the teachings of the present invention may also be used in recombinant production of polypeptides which are native to the parental cells. Examples of such use include, but are not limited to, placing a gene encoding the polypeptide under the control of a different promoter to enhance expression of the polypeptide, to expedite export of a native polypeptide of interest outside the cell by use of a signal sequence, or to increase the copy number of a gene encoding the protein normally produced by the subject parental cells. Thus, the present invention also encompasses recombinant production of homologous polypeptides, to the extent that such expression involves the use of genetic elements not native to the parental cell, or use of native elements which have been manipulated to function in a manner not normally expected in the parental cell.

The improved strains of filamentous fungus are assayed for their total protein of interest production and/or the activity of the protein of interest that is being over-expressed. Such improved strains of filamentous fungus are characterized and tested according to assays known to those skilled in the art. For example, the improved strains are evaluated for their ability to secrete cellulase by known cellulase enzyme assays. An improved strain is obtained if the total cellulase production and/or activity of cellulase is higher than a strain with similar genetic background which has not been subject to the method of the present invention.

In various embodiments, under identical conditions, candidate cells can produce at least about 0.1%, 0.2%, 0.5%, 1%, 2%, 2.5%, 5%, 7.5%, 10%, 15%, 20%, 25%, 30%, 40%, 50%, 100%, 200%, 400%, 600%, 800%, or 1000% more cellulase, or another polypeptide(s) of interest, than the starting parental cells and the test cells that were not contacted with a mutagen.

The present invention may be better understood by reference to the following non-limiting examples, which are provided only as exemplary of the invention. The following examples are presented to more fully illustrate the preferred embodiments of the invention. The examples should in no way be construed, however, as limiting the broader scope of the invention.

### 8. EXAMPLES

### 8.1. Materials

50× Vogels Stock solution was composed of 750 mL dH₂O per liter, 125 g of Na₃Citrate 2H₂O; 250 g of KH₂PO₄ (Anhydrous); 100 g of NH₄NO₃ (Anhydrous); 10 g of MgSO₄ 7H₂O; 5 g of CaCl₂ 2H₂O; 5 ml of Vogels Trace Element Solution; 2.5 ml of Vogels Biotin Solution; and bring to volume with dH₂O (see Davis et al., 1970, Methods in Enzymology 17A, pg 79-143; and Davis et al., 2000, Neurospora, Contributions of a Model Organism, Oxford University Press, for information on Vogels minimal medium).

Vogels trace elements solution contained in 1 liter of dH₂O, 50 g of Citric Acid; 50 g of ZnSO₄ 7H₂O; 10 g of Fe(NH₄)₂SO₄ 6H₂O; 2.5 g of CuSO₄ 5H₂O; 0.5 g of MnSO₄ 4H₂O; 0.5 g of H₃BO₃ (Boric Acid); and 0.5 g of NaMoO4 2H₂O.

Vogels biotin solution comprises 0.1 g of d-Biotin in 1 liter.

Vogels plates formula includes 20 ml of 50× Vogels stock solution, 20 g of BBL Agar (not EM), 975 ml of dH₂O. The solution was autoclaved and after sterilization, 25 ml of 40% Glucose (or 20 ml 50% Glucose) is added per liter.

### 8.2. Constructing Test Cells

Trichoderma reesei RL-P37 was obtained from the American Type Culture Collection (Maryland, USA). RL-P37L was created by transforming RL-P37 with a laccase from Stachybotrys chartarum MUCL38898. The laccase was expressed using the T. reesei cellulase cbh1 promoter (see Amory, A. et al., 1999. International patent, WO99/49020).

An expression plasmid for use in transforming Trichoderma reesei is constructed as follows. The gene encoding a laccase was cloned from Stachybotrys chartarum MUCL38898 by PCR as described in Amory, A. et al. 1999, WO99/49020, and inserted into a PstI site of the Trichoderma expression vector, pTrex (see, e.g., U.S. Pat. No. 6,426,410).

The expression cassette within the vector contains a cbh1 promoter upstream of the PstI site, a 1.25 kb terminator down stream of the PstI site. A second 1.3 kb CBH1 terminator is downstream of the pyr 4 selectable marker. The expression cassette was removed from the pTrex vector using NotI digest and inserted into the Agrobacterium pPZP100 vector (see Hajdukiewiez et al., 1994, Plant Molecular Biology 25, 989-994). This vector contains the border sequences which enables Agrobacterium tumefaciens-mediated transformation. The PZP 100 vector containing the laccase expression cassette was transformed into Agrobacterium tumefaciens EHA 101 competent cells (see Hood et al., 1986, Journal of Bacteriology 168, 1291-1301).

For Agrobacterium tumefaciens-mediated transformation of Trichoderma reesei conidia, 10⁵ to 10⁷ conidia was mixed with about 10⁹ A. tumefaciens cells and the mixtures were plated on nitrocellulose filters placed on IM plates containing 5 mM glucose and 0.25 mg of uridine. The plates were incubated at room temperature for 1 day. Hereafter, the filters were transferred to Vogels minimal medium plates.

Transformants were plated onto Vogels agar containing 5 mM ABTS. A transformant, RL-P37L showing the lowest amount of laccase production based on color on Vogels/ABTS plates was selected for strain improvement.

The methylating compound N-methyl-N′-nitro-N-nitrosoguanidine (NTG) is one of the most potent mutagens available. It induces primarily base transition mutations of the GC to AT type (although AT to GC transitions, transversions, and frameshifts arise at low frequencies). To generate genetic diversity, RL-P37L spores were mutated using NTG until a kill of 50% was obtained (see Eisenstadt, E., Carlton, B., Brown, B. 1994. Gene Mutation. In, “Methods for General and Molecular bacteriology”. Gerhardt, P., Murray, R. G., Wood, W., and Krieg, N. eds. American Society for Microbiology: Washington, D.C. pp. 297-316). The resulting mutants were screened for laccase production under repressible conditions.

Fresh fungal plates were prepared and used to obtain a spore suspension containing about 1×10⁹ spore forming units/ml. The number of spore forming units/ml was determined using a hemocytometer. A solution of NTG (Aldrich-4991) was freshly prepared to a concentration of 15 mg/mL in DMSO and added at a final concentration of 1.0 mg/ml to the fungal spore suspension. The fungal spore suspension was then incubated at room temperature in the dark until the desired % kill is obtained.

The first time a strain is mutated a kill curve is prepared. Starting at time zero, samples are taken every 30 minutes and a viable spore count is conducted. Once the kill curve is established, only the time zero and the final viable count are made to ensure the correct % kill has been obtained. For RLP37L, the spores were incubated with 1 mg/mL NTG for 30 minutes to obtain a 50% kill. After incubation, the NTG is removed by washing the spores at least 3× in water. Aliquots are prepared of the mutated spores and they are stored in glycerol at −70° C.

### 8.3 Screening Assay

For the primary screen, mutant cells that were derepressed in the presence of glucose were isolated by the expression of laccase as a reporter. Mutant spores were plated on Vogels agar plate medium containing 20% glucose at a density of 10⁵ and the plates were cultured at 28° C. for 4 days. Expression of laccase in T. reesei mutant cells was detected by a green color generated by the oxidation of the laccase substrate 2,2′-azinobis-(3-ethybenzthiazoline-6-sulfonic acid) (“ABTS”) which was present at 5 mM in the agar medium. (see Davis et al., 1970, Methods Enzymol. 17, 79-143).

Titration experiments were done using glucose concentrations from 1 to 20%. A concentration greater than 10% was used in order to turn off laccase expression in the majority of variants. These lower levels can be used when individual colonies can be viewed on the agar plate. For high through-put screening, 20% glucose was found to be optimal as 99.9% of mutants did not produce laccase.

A secondary screen was carried to determine protein production and cellulase activity. The total amount of protein produced by the mutant cells was determined using a BCA total protein assay kit (Pierce). For cellulase activity, acid swollen cellulose (“ASC”) was used as a substrate. ASC was made by preparing a slurry of 25 g of AVICEL™ (FMC Corp., Pennsylvania) in 100 ml of water to which about 20 ml of 85% phosphoric acid was added. To this, 1 kg of 85% phosphoric acid was added, and the solution was mixed. The mixture was then slowly poured into 5 liters of water resulting in the precipitation of the ASC. The ASC was collected by filtering through mira cloth (Calbiochem-Nova, California, USA) and washed with 3 liters of water. Finally, the ASC was mixed in a blender until smooth. The secondary screening medium was Vogels medium (50× Vogels medium was composed per liter of 150 g of sodium citrate, 250 g of KH₂ PO₄, 10 g of MgSO₄.7H₂O, 10 g of CaCl₂.2H₂O, 2.5 ml of biotin stock solution, and 5.0 ml of AMG trace metals solution) containing 5% ASC. Liquid culture shake flask medium was used (see Ilmen et al., 1997, App Environ Microbiol 63, 1298-1306), except that 100 mM piperazine-N,N′-bis(2-ethanesulfonic acid) (PIPES; Calbiochem) was included to maintain the pH at 5.5.

### 8.4 Results

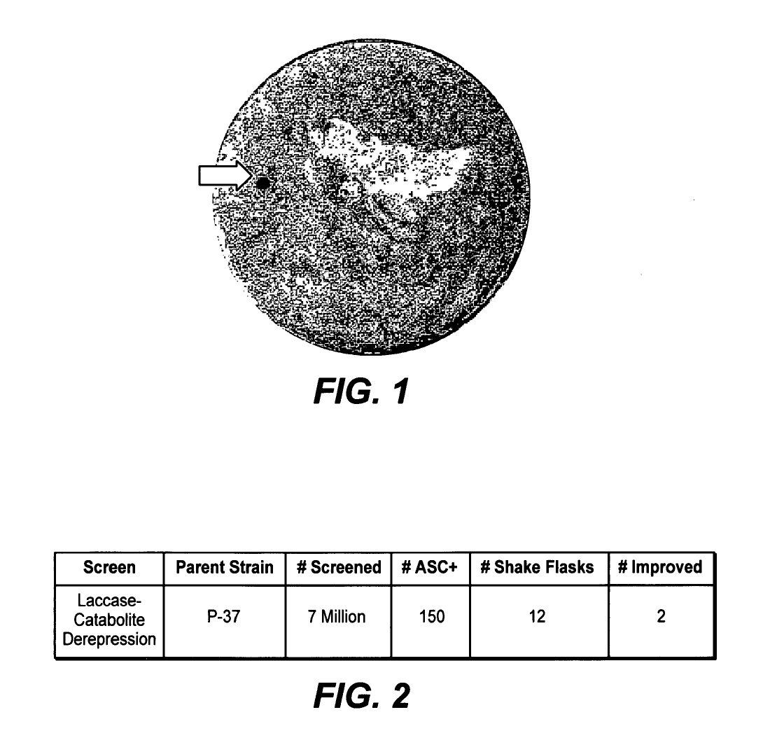

Using the primary screen, about 1 million NTG-mutated T. reesei spores were screened. Mutants cells were cultured under catabolite repressible conditions and candidate cells that produced laccase were isolated. A positive mutant can be visually identified among 100,000 mutated spores when grown in the presence of 20% glucose and the laccase substrate 5 mM ABTS on a 150×15 mm Vogel agar plate. See FIG. 1.

By transforming cells with Agrobacterium tumefaciens, one copy of the reporter gene is inserted, thereby providing for more effective detection of improved fungal strains because the background level of the reporter is kept low. If other methods are used wherein the background reporter levels are high, the screen may work less effectively because ABTS produced can diffuse from the colony, making it difficult to identify the colony that is producing the ABTS activity.

FIG. 2 shows the screening efficiency of primary screens. The number of spores screened and subsequent transfers to acid swollen cellulase plates and to shake flasks for evaluation is shown. The number of strains showing improved cellulase productivity in shake flask compared to the parent was determined for each primary screen. Improved strains were further evaluated in 14 L fermentors under commercial production conditions.

About 150 candidate cells that produced laccase were subsequently isolated and screened for improved protein production and cellulase production in the secondary screen. Mutant cells formed colonies on ASC-containing medium and produced clearing zones or halos where the ASC is digested. The halo-to-colony size ratio of discrete colonies was measured. Higher ratios were indicative of improved cellulase production.

Twelve strains were selected for further study in shake flasks. The results are shown in Table 1 below. Two mutants, LC5 and LC 36, were found to produce 15% and 20% more total secreted protein compared to the RL-P-37 control, respectively (RL-P-37 is the parent; the LC strains are the improved strains).

TABLE 1
Production of total protein in shake flasks. Results of RL-P-37 NTG
mutants isolated for catabolite derepression and over-expression
of cellulase on agar plates.

| | Total BCA protein | Clearing on ASC |
|---|---|---|
| Strains | g/L +/− S.D. | Colony size/clearing zone |
| RL-P-37 | 837 +/− 48 | 0.57 |
| LC1 | 800 +/− 32 | 0.55 |
| LC3 | 772 +/− 28 | 0.61 |
| LC4 | 852 +/− 52 | 0.59 |
| LC5 | 982 +/− 55 | 0.45 |
| LC7 | 755 +/− 33 | 0.6 |
| LC14 | 688 +/− 43 | 0.67 |
| LC17 | 841 +/− 62 | 0.59 |
| LC23 | 828 +/− 24 | 0.59 |
| LC31 | 769 +/− 61 | 0.62 |
| LC36 | 1036 +/− 45 | 0.43 |
| LC43 | 854 +/− 28 | 0.56 |
| LC46 | 776 +/− 52 | 0.64 |

When these two strains were tested for cellulase activity, LC5 and LC36 produced 25% and 30% higher cellulase activities than RL-P-37 respectively, as determined in a filter paper assay (see Table 2). Substrates for the cellulytic enzymes included Whatman no. 1 filter paper strips (1 by 6 cm [50 mg]) for filter paper activity. The production of free reducing sugar was quantitated by the dinitrosalicyclic acid method. Activity towards filter paper is based on the release of 0.5 to 1.0 mg of reducing sugar/ml of the 50 mg filter paper strips. (see Montenecourt et al., Appl. Environ. Microbiol. 1997, 34, 777-782).

TABLE 2
LC5 and LC36 strains produce higher cellulase activities.

| Strains | Filter paper U/ml | Total BCA protein mg/L |
|---|---|---|
| RL-P-37 | 9.0 | 763 |
| LC5 | 12 | 910 |
| LC36 | 13 | 1076 |

### 9. EQUIVALENTS

The present invention is not to be limited in scope by the specific embodiments described which are intended as single illustrations of individual aspects of the invention, and functionally equivalent methods and components are within the scope of the invention. Indeed, various modifications of the invention, in addition to those shown and described herein, will become apparent to those skilled in the art from the foregoing description and accompanying drawings using no more than routine experimentation. Such modifications and equivalents are intended to fall within the scope of the appended claims.

All publications, patents and patent applications mentioned in this specification are herein incorporated by reference into the specification to the same extent as if each individual publication, patent or patent application was specifically and individually indicated to be incorporated herein by reference.

Citation or discussion of a reference herein shall not be construed as an admission that such is prior art to the present invention.

1. A method for obtaining an improved strain of filamentous fungus, comprising:
contacting test cells of a strain of filamentous fungus with a mutagen thereby producing a population of mutant cells, wherein said test cells comprise a reporter gene construct and said reporter gene construct comprises a promoter operably linked with a reporter gene;
culturing said population of mutant cells under conditions that repress the activity of the promoter; and
isolating cells that produce the reporter from said population of mutant cells.

2. A method for obtaining an improved strain of filamentous fungus, comprising:
providing test cells of a strain of filamentous fungus, said test cells comprising a reporter gene construct;
contacting said test cells with a mutagen thereby producing a population of mutant cells, wherein said reporter gene construct comprises a promoter operably linked with a reporter gene;
culturing said population of mutant cells under conditions that repress the activity of the promoter; and
isolating cells that produce the reporter from said population of mutant cells.

3. The method of claim 1 or 2, further comprising determining the level of a polypeptide of interest produced by said isolated cells.

4. The method of claim 1 or 2, wherein the filamentous fungus is a species of Acremonium, Aspergillus, Chrysosporium, Fusarium, Gliocladium, Humicola, Myceliophthora, Mucor, Neurospora, Penicillium, Thielavia, Tolypocladium, Trichoderma, or teleomorphs or synonyms thereof.

5. The method of claim 4, wherein the filamentous fungus is Trichoderma reesei, Trichoderma viride, Trichoderma longibrachiatum, Trichoderma harzianum, or Trichoderma koningii.

6. The method of claim 1 or 2, wherein the reporter is an enzyme.

7. The method of claim 6, wherein the reporter is a fungal laccase.

8. The method of claim 7, wherein the fungal laccase is a laccase of a species of Acremonium, Agaricus, Antrodiella, Armillaria, Aspergillus, Aureobasidium, Bjerkandera, Cerrena, Chaetomium, Chrysosporium, Cryptococcus, Cryphonectria, Coprinus, Curvularia, Cyathus, Daedalea, Filibasidium, Fomes, Fusarium, Geotrichum, Halosarpheia, Humicola, Lactarius, Lentinus, Magnaporthe, Monilia, Monociium, Mucor, Myceliophthora, Neocallimastix, Neurospora, Paecilomyces, Panus, Penicillium, Phanerochaete, Phellinus, Phlebia, Pholiota, Piromyces, Pleurotus, Podospora, Pycnoporus, Pyricularia, Rhizoctonia, Rigidoporus, Schizophyllum, Sclerotium, Scytalidium, Sordaria, Sporotrichum, Stagonospora, Talaromyces, Thermoascus, Thielavia, Tolypocladium, Trametes, and Trichoderma.

9. The method of claim 7, wherein the reporter is laccase of a Stachybotrys species.

10. The method of claim 1 or 2, wherein the mutagen is ultra violet light, X-ray, gamma radiation, nitrous acid, nitrosamines, nitrosoguanidine, methyl nitrosoguanidine, 5-bromouracil, restriction enzyme-mediated integration, or any combination thereof.

11. The method of claim 1 or 2, wherein the promoter is a catabolite repressible promoter.

12. The method of claim 1 or 2, wherein the promoter is a temperature-sensitive promoter.

13. The method of claim 1 or 2, wherein the promoter is regulated by changes in osmolarity.

14. The method of claim 1 or 2, wherein the promoter is a promoter that regulates the expression of cbh1, cbh2, eg1, eg2, eg3, eg5, xln1, or xln2 in Trichoderma species.

15. The method of claim 1 or 2, wherein the conditions that repress the activity of the promoter is pH, temperature, osmolarity, the presence of an inhibitor, the concentration of an inhibitor, or a combination of any two or more of the foregoing.

16. The method of claim 1 or 2, wherein said isolated cells produce at least about 10%, or at least about 20%, or at least about 30% more cellulase enzymes than the test cells that were not contacted with said mutagen.

17. The improved strain of filamentous fungus made by the method according to claim 1 or 2.

18. An improved strain of filamentous fungus, wherein cells of said strain comprise a reporter gene construct that comprises a promoter operably linked with a reporter gene, and wherein said cells produce the reporter under conditions that repress activity of the promoter, and wherein said cells are made by:
contacting cells of said strain with a mutagen and isolating surviving cells that produce a detectable amount of the reporter under conditions that repress activity of the promoter.
